(19) United States
(12) Patent Application Publication
(10) Pub. No.: US 2008/0243122 A1
(43) Pub. Date: Oct. 2, 2008

(54) APPARATUSES AND METHODS FOR BONE SCREW AUGMENTATION

(76) Inventors: Andrew C. Kohm, Burlingame, CA (US); Arthur E. Ferdinand, San Jose, CA (US)

Correspondence Address: COOLEY GODWARD KRONISH LLP;ATTN: PATENT GROUP, Suite 1100, 777 - 6th Street, NW, WASHINGTON, DC, 20001, US

(21) Appl. No.: 11/693,180

(22) Filed: Mar. 29, 2007

(51) Int. Cl. A61B 17/00; A61B 17/56; A61M 29/00

(52) U.S. Cl. 606/80; 606/191

(57) ABSTRACT

An apparatus includes an expandable member and a cutting member coupled to the expandable member. The expandable member is configured to form a cavity within a cancellous portion of a bone. The cutting member is configured to cut a cortical portion of the bone.

### BACKGROUND

The invention relates generally to medical devices and procedures, and more particularly to medical devices and procedures for augmenting bone screws.

Bone screws are often used to stabilize bone structures within the body. For example, some known bone screws can be used as part of a fracture repair procedure. Known bone screws can also be used with other structural members, such as, for example, rods, bars and/or plates as part of a spinal fixation procedure. Such known bone screws, which are configured to be implanted into the vertebral pedicle, are often referred to as pedicle screws.

Once implanted into a host bone structure, some known bone screws can be susceptible to becoming “pulled out” from the host bone structure, loosened within the host bone structure and/or destabilized within the host bone structure. The susceptibility of a bone screw to pullout, loosening and/or destabilization can be compounded when the bone screw is implanted into soft portions of a bone structure (e.g., cancellous bone or diseased bone) and/or the bone screw is subjected to high loads. Accordingly, in certain situations bone cement can be used to augment the pull-out strength of known bone screws. The use of bone cement to augment a known bone screw, however, can be difficult and may not, in fact, increase the pull-out strength of the bone screw.

Thus, a need exists for improved apparatuses and methods for augmenting bone screws.

### SUMMARY

Apparatuses and methods for augmenting bone screws are described herein. In some embodiments, an apparatus includes an expandable member and a cutting member coupled to the expandable member. The expandable member is configured to form a cavity within a cancellous portion of a bone. The cutting member is configured to cut a cortical portion of the bone.

### BRIEF DESCRIPTION OF THE DRAWINGS

FIG. 1 is a flow chart illustrating the method of augmenting a fixation device according to an embodiment of the invention.

FIGS. 2-4 are schematic illustrations showing a bone structure in a first configuration, a second configuration and a third configuration, respectively, according to the method illustrated in FIG. 1.

FIG. 5 is a cross-sectional side view from a transverse plane of a vertebra having a first and a second cavity formed by a method according to an embodiment of the invention.

FIG. 6 is a cross-sectional side view from a transverse plane of a medical device according to an embodiment of the invention disposed within the vertebra shown in FIG. 5.

FIG. 7 is a close-up view of the medical device shown in FIG. 6.

FIG. 8 is a side view of a medical device for forming a cavity within a bone structure according to an embodiment of the invention.

FIG. 9 is a side view of a medical device for forming a cavity within a bone structure according to an embodiment of the invention.

FIG. 10 is a side view of a medical device for forming a cavity within a bone structure according to an embodiment of the invention.

FIG. 11 is a cross-sectional side view from a transverse plane of a vertebra having a first and a second cavity formed by a method according to an embodiment of the invention.

FIG. 12 is a cross-sectional side view from a transverse plane of a medical device according to an embodiment of the invention disposed within the vertebra shown in FIG. 11.

FIG. 13 is a close-up view of the medical device shown in FIG. 12.

FIG. 14 is a cross-sectional side view from a transverse plane of a vertebra having a first and a second cavity formed by a method according to an embodiment of the invention.

FIG. 15 is a cross-sectional side view from a transverse plane of a medical device according to an embodiment of the invention disposed within the vertebra shown in FIG. 14.

FIG. 16 is a close-up view of the medical device shown in FIG. 15.

FIG. 17 is a cross-sectional view of the medical device taken along line 17-17 shown in FIG. 16.

FIG. 18 is a cross-sectional side view from a transverse plane of a vertebra having a first, a second cavity and a third cavity formed by a method according to an embodiment of the invention.

### DETAILED DESCRIPTION

In some embodiments, an apparatus includes an expandable member and a cutting member coupled to the expandable member. The expandable member, which can be, for example, an inflatable member, is configured to form a cavity within a cancellous portion of a bone. The cutting member, which can be, for example, a whisk, a curette or the like, is configured to cut a cortical portion of the bone.

In some embodiments, an apparatus includes a first shaft, a second shaft, an expandable member and a cutting member coupled to the expandable member. The expandable member is configured to form a cavity within a cancellous portion of a bone. The cutting member is configured to cut a cortical portion of the bone. A portion of the second shaft is rotatably disposable within the first shaft. A portion of the cutting member is coupled to an outer surface of the second shaft such that the cutting member produces a cutting force on the cortical portion of the bone of when the second shaft is rotated within the first shaft and the cutting member is in contact with the cortical portion of the bone.

In some embodiments, a method includes forming a first cavity within a first portion of a bone. A second cavity is formed within a second portion of the bone, the first portion and the second portion being mutually exclusive. A material, such as for example, bone cement, is conveyed to the first cavity and the second cavity. A fixation device, such as for example, a pedicle screw, is inserted into the bone such that a first portion of the fixation device is disposed within the first cavity and in contact with the material, and a second portion of the fixation device is disposed within the second cavity and in contact with the material.

In some embodiments, a method includes forming a first cavity within a first portion of a bone by removing a portion of the first portion of the bone. A second cavity is formed within a second portion of the bone by compressing a portion of the second portion of the bone. The first portion of the bone and the second portion are mutually exclusive. In some embodiments, a shape of the second cavity is different than a shape of the first cavity. A material, such as for example, bone cement, is conveyed to the first cavity and the second cavity. A fixation device is inserted into the bone such that a first portion of the fixation device is disposed within the first cavity and in contact with the material, and a second portion of the fixation device is disposed within the second cavity and in contact with the material.

In some embodiments, an apparatus includes an anchoring member configured to limit the movement of a pedicle screw within a vertebra. A distal portion of the anchoring member has a size greater than a size of a proximal portion of the anchoring member.

In some embodiments, an apparatus includes an anchoring member configured to limit the movement of a pedicle screw within a vertebra. A distal portion of the anchoring member has a size greater than a size of a proximal portion of the anchoring member. The anchoring member has a first configuration in which a portion of the anchoring member is substantially liquid and a second configuration in which the portion of the anchoring member is substantially solid. The anchoring member is configured to limit the movement of the pedicle screw when in the second configuration.

In some embodiments, a kit includes a catheter and a cannula. The catheter has an expandable member and a cutting member disposed proximate to the expandable member. The expandable member is configured to form a cavity within a cancellous portion of a bone. The cutting member is configured to cut a cortical portion of the bone. The cannula, which is configured to receive a portion of the catheter, is configured be inserted percutaneously.

In some embodiments, an apparatus includes a portion of a cortical bone, a portion of a cancellous bone, an anchoring member and a fastener. The portion of the cortical bone defines a first cavity. The portion of a cancellous bone defines a second cavity that is displaced from the first cavity along a longitudinal axis. The anchoring member has a first portion and a second portion. The first portion of the anchoring member is disposed within the first cavity. The second portion of the anchoring member is disposed within the second cavity. The fastener is coupled to the anchoring member.

As used in this specification and the appended claims, the singular forms “a,” “an” and “the” include plural referents unless the context clearly dictates otherwise. Thus, for example, the term “a member” is intended to mean a single member or a combination of members, “a material” is intended to mean one or more materials, or a combination thereof. Furthermore, the words “proximal” and “distal” refer to direction closer to and away from, respectively, an operator (e.g., surgeon, physician, nurse, technician, etc.) who would insert the medical device into the patient, with the tip-end (i.e., distal end) of the device inserted inside a patient's body first. Thus, for example, the end of the expandable member first inserted inside the patient's body would be the distal end of the expandable member, while the end of the expandable member to last enter the patient's body would be the proximal end of the expandable member.

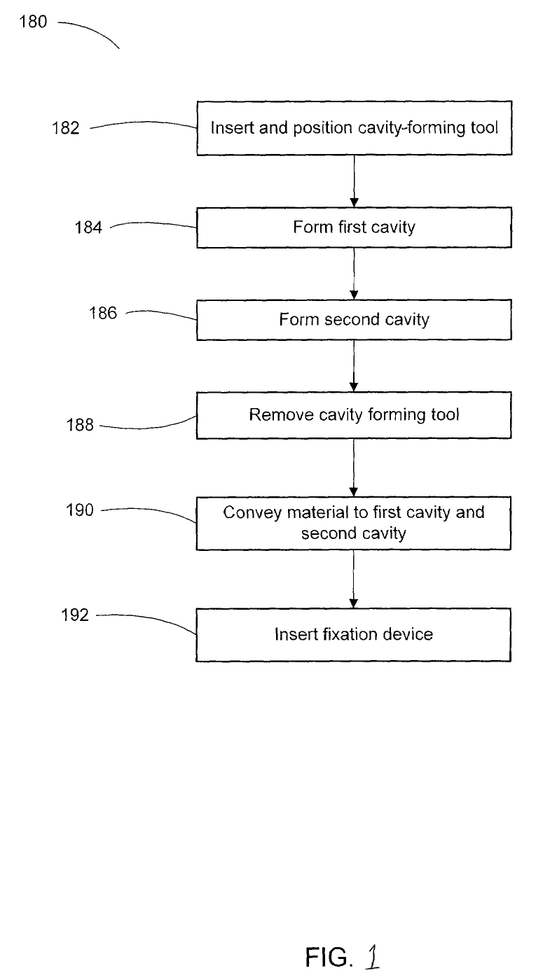

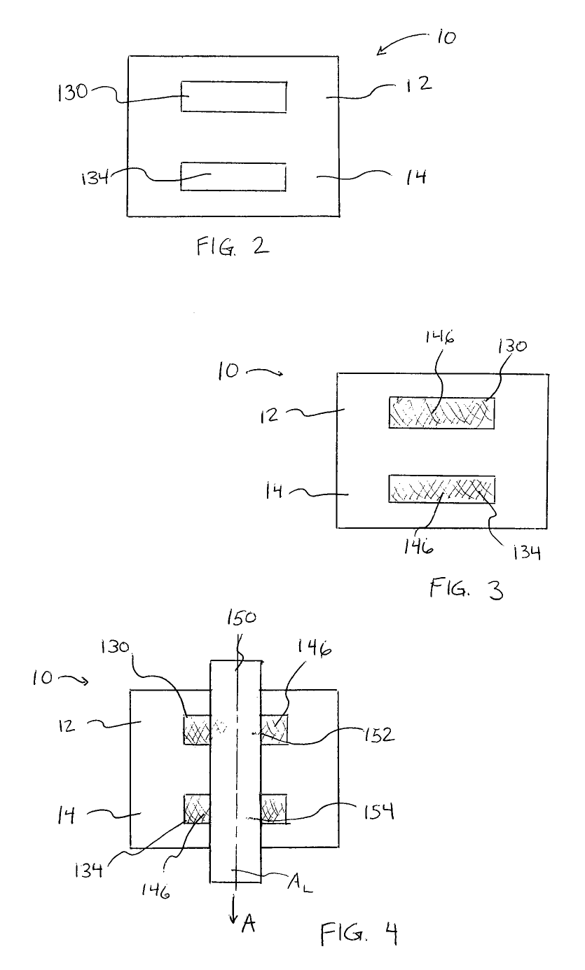

FIG. 1 is a flow chart illustrating a method 180 of augmenting a fixation device according to an embodiment of the invention. The illustrated method is described with reference to FIGS. 2-4, which are schematic illustrations showing a bone structure in a first configuration, a second configuration and a third configuration, respectively. The illustrated method includes inserting and positioning a cavity-forming tool. The cavity-forming tool is configured to form a cavity within a bone structure and/or bodily tissue, 182. In some embodiments, the bone structure can be, for example, a vertebra. The cavity-forming tool (not shown in FIGS. 2-4) can be, for example, a catheter having an expandable member and/or a cutting member. In some embodiments, the cavity-forming tool can be inserted percutaneously. Details of the cavity-forming tool are discussed in more detail herein.

The illustrated method then includes forming a first cavity within a first portion of the bone structure at 184. Similarly, the method includes forming a second cavity within a second portion of the bone structure at 186. The first portion of the bone structure and the second portion of the bone structure are mutually exclusive. Referring to FIG. 2, which shows a bone structure 10 in a first configuration, the first cavity 130 is formed within the first portion 12 of the bone structure 10. Similarly, the second cavity 134 is formed within the second portion 14 of the bone structure 10. Although the first cavity 130 is shown as being spaced apart from the second cavity 134, in other embodiments, the first cavity 130 can be contiguous (i.e., can share a common boundary) with the second cavity 134.

Returning to the flow chart shown in FIG. 1, the method then includes removing the cavity-forming tool, 188, and conveying a material to the first cavity and the second cavity, 190. For example, FIG. 3 shows the bone structure 10 in a second configuration, in which the material 146 has been conveyed to the first cavity 130 and the second cavity 134. The material 146 can be, for example, any material configured to augment a fixation device. In some embodiments, for example, the material 146 can be a bone cement configured to be conveyed to the first cavity 130 and/or the second cavity 134 in a substantially liquid state and to harden into a substantially solid state after being conveyed to the first cavity 130 and/or the second cavity 134. Such a bone cement can include, for example, compositions containing polymethylmethacrylate (PMMA), barium sulfate, hydroxyapatite, monocalcium phosphate, calcium carbonate, bone chips or the like. In other embodiments, the material 146 can configured to be conveyed to the first cavity 130 and/or the second cavity 134 in a first state, in which the material has a low viscosity and to subsequently cure into a second state, in which the material has a higher viscosity. In some embodiments, the material 146 can be a bone cement of the type shown and described in International Patent Application Publication No. WO2006/090379 A2, entitled “Methods, Materials and Apparatus for Treating Bone and Other Tissue,” filed on Feb. 22, 2006, and incorporated herein by reference in its entirety. In some embodiments, the material 146 can be conveyed to the first cavity 130 and/or the second cavity 134 percutaneously via a cannula.

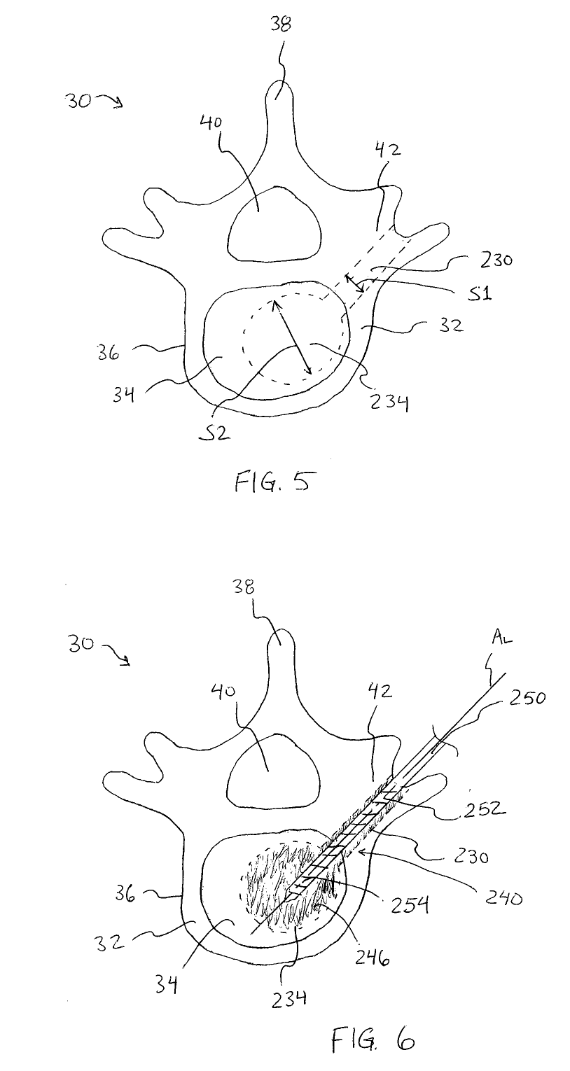

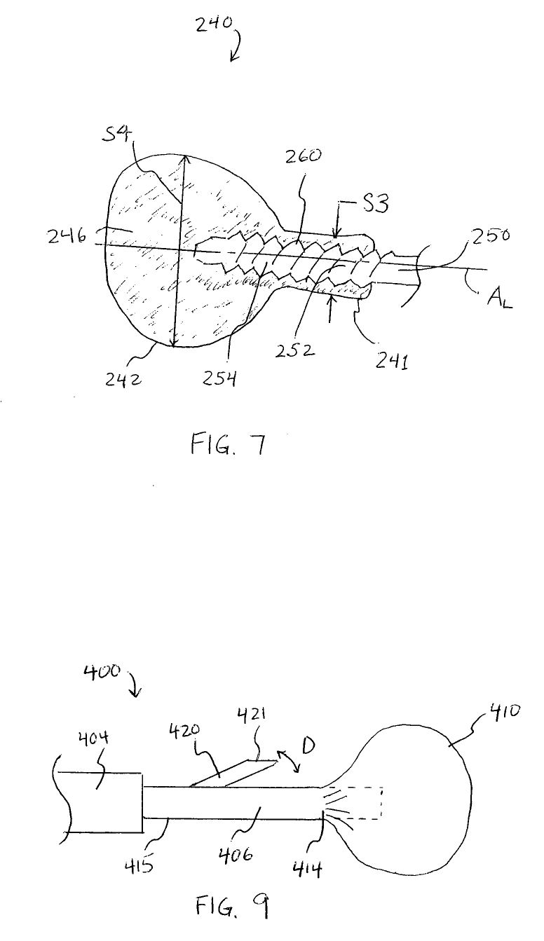

Returning to the flow chart shown in FIG. 1, the method then includes inserting a fixation device into the bone structure at 192 such that a first portion of the fixation device is disposed within the first cavity and in contact with the material, and a second portion of the fixation device is disposed within the second cavity and in contact with the material. This operation is shown schematically in FIG. 4, which shows a fixation device 150 being inserted into the bone structure 10, as indicated by the arrow A. The fixation device 150, which can be, for example, a bone screw, a pedicle screw, a fixation rod, a plate or the like, has a first portion 152 and a second portion 154. As illustrated in FIG. 4, the first portion 152 is disposed within the first cavity 130 and in contact with the material 146. Similarly, the second portion 154 is disposed within the second cavity 134 and in contact with the material 146. In some embodiments, for example, the fixation device 150 can be inserted percutaneously via a cannula.

In this manner, the fixation device 150 can be augmented within the bone structure 10 by the material 146. Said another way, in some embodiments, the material 146 can correspond to the shape of and anchor within the first cavity 152 and/or the second cavity 154 to increase the strength of the fixation device 150 within the bone structure 10. Moreover, because the material 146 is in contact with mutually exclusive portions of the fixation device 150 (e.g., the first portion 152 and the second portion 154), the fixation device 150 can be augmented differently by the material 146 within the first cavity 152 and the material 146 within the second cavity 154. In some embodiments, for example, the material 146, the first cavity 152 and/or the second cavity 154 can be configured to increase the pull-out strength of the fixation device when disposed within the bone structure 10 (e.g., to increase the resistance of the fixation device 150 from being moved along its longitudinal axis A_{L}). In other embodiments, the material 146, the first cavity 152 and/or the second cavity 154 can be configured to increase the resistance of the fixation device 150 to being rotated about its longitudinal axis A_{L} and/or any other axis defined by the fixation device 150 when disposed within the bone structure 10. In yet other embodiments, the material 146, the first cavity 152 and/or the second cavity 154 can be configured to reduce the likelihood of the fixation device 150 loosening and/or becoming destabilized when disposed within the bone structure 10.

In some embodiments, the first portion 12 of the bone structure 10 can have different characteristics than the second portion 14 of the bone structure 10. For example, in some embodiments, the first portion 12 of the bone structure 10 can be a hard, dense structure, such as cortical bone, and the second portion 14 of the bone structure can be a softer, less dense structure, such as cancellous bone. Accordingly, in some embodiments, the first cavity 130 can be configured to augment the fixation device 150 in a certain way (i.e., to increase its resistance to rotation about the longitudinal axis A_{L}) and the second cavity 134 can be configured to augment the fixation device 152 in another way (i.e., to increase its resistance to linear movement along the longitudinal axis A_{L}).

Similarly, because of the different characteristics of the first portion 12 of the bone structure 10 and the second portion 14 of the bone structure 10, in some embodiments, the first cavity 130 can be formed by different methods than the second cavity 134. For example, in some embodiments, the first cavity 130 can be formed by removing a portion of the first portion 12 of the bone structure 10 and the second cavity 134 can be formed by compressing a portion of the second portion 14 of the bone structure 10.

Although the first cavity 130 and the second cavity 134 are shown as having similar sizes and/or shapes, in some embodiments, the first cavity 130 and the second cavity 134 can have different sizes and/or shapes. In this manner, the shape and/or size of the cavities can be configured to ensure that the fixation device is augmented as desired. For example, FIG. 5 is a cross-sectional side view from a transverse plane of a vertebra 30 in a first configuration defining a first cavity 230 and a second cavity 234 having different sizes and shapes. FIG. 6 is a cross-sectional side view of the vertebra 30 in a second configuration, in which an anchoring member 240 according to an embodiment of the invention is disposed. The vertebra 30 includes a spinous process 38, a spinal cord canal 40, a pedicle 42 and a vertebral body 36. The vertebral body 36 includes an outer portion 32 formed from a cortical bone surrounding an inner portion 34 formed from cancellous bone. The first cavity 230 is defined by at least a portion of the pedicle 42 and/or at least a portion of the outer portion 32 of the vertebral body 36 and has a substantially cylindrical shape characterized by a size S1 (e.g., the diameter of the cylinder characterizing the first cavity 230). Similarly, the second cavity 234 is defined by at least a portion of the inner portion 34 of the vertebral body 36 and has a substantially spherical shape characterized by a size S2 (e.g., the diameter of the sphere characterizing the first cavity 234), which is larger than the size S1.

As shown in FIG. 6, the anchoring member 240 includes a material 246, which can be conveyed into the first cavity 230 and the second cavity 234 as described above, and a fixation device 250. The fixation device 250 is disposed within the vertebra 30 such that a first portion 252 of the fixation device 250 is disposed within the first cavity 230 and in contact with the material 246 contained therein. Similarly, a second portion 254 of the fixation device 250 is disposed within the second cavity 234 and in contact with the material 246 contained therein. In this manner, when the material 246 is in a substantially solid state, the material 246, the first portion 252 of the fixation device 250 and the second portion 254 of the fixation device 250 form the anchoring member 240.

As shown in FIG. 7, the anchoring member 240 has a proximal end portion 241 and a distal end portion 242. The proximal end portion 241 has a substantially cylindrical shape corresponding to the shape of the first cavity 230 having a size S3 (e.g., the diameter of the cylinder characterizing the proximal end portion 241). Similarly, the distal end portion 242 has a substantially spherical shape corresponding to the shape of the second cavity 234 having a size S4 (e.g., the diameter of the sphere characterizing the distal end portion 242). Because the size S4 of the distal end portion 242 is larger than the size S3 of the proximal end portion 241, the anchoring member 240, and therefore the fixation device 250, is resistant to being pulled out of the vertebra 30 (e.g., the fixation device 250 is resistant to being moved along the longitudinal axis A_{L}). Moreover, the fixation device 250 can include a series of threads 260 to provide recesses in which the material 246 can flow when in a liquid state, thereby augmenting the strength of the fixation device 250 (e.g., the pull-out strength, the resistance to being rotated, the resistance to be loosened and/or the resistance to becoming destabilized) when the material 246 is in a solid state.

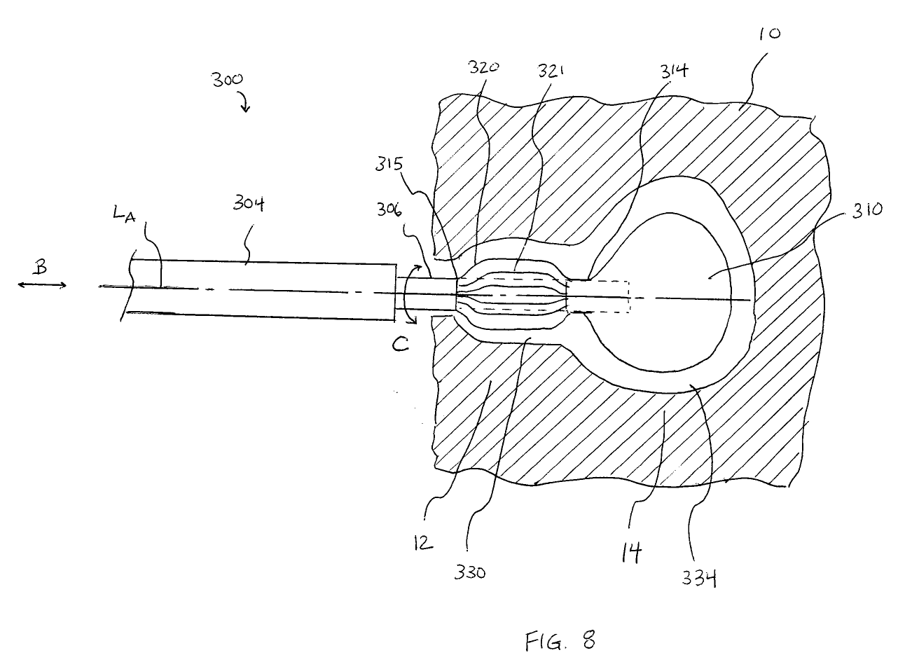

FIG. 8 shows a portion of a catheter assembly 300 according to an embodiment of the invention configured to form cavities within bone and/or human tissue, as described above. The catheter assembly 300 includes a first shaft 304, which can be, for example, a cannula, and a second shaft 306 movably disposed within the first shaft 304. An expandable member 310 is coupled to a distal end portion 314 of the second shaft 306. In some embodiments, the expandable member 310 can be a low-compliant balloon of the type shown and described in U.S. patent application Ser. No. 11/438,693, entitled “Low-Compliance Expandable Medical Device,” filed on May 23, 2006 and U.S. Provisional Patent Application Ser. No. 60/884,050, entitled, “Apparatus and Methods for use of Expandable Members in Surgical Application,” filed on Jan. 9, 2007, each of which is incorporated herein by reference in its entirety. A cutting member 320 is coupled to an outer surface 315 of the second shaft 306 proximate to the expandable member 310. In some embodiments, the cutting member 320 can be configured to collapse or retract to be disposed within the first shaft 304 when not in use. For example, in some embodiments, the cutting member 320 can be a metallic whisk that includes multiple cutting elements 321, each of which can be retracted to be disposed within the first shaft 304 and to be in contact with the outer surface 315 of the second shaft 306. In some embodiments, the cutting member 320 can be a metallic whisk that includes multiple cutting elements 321 that can be actuated to extend and retract independent from being disposed within the first shaft 304. In some embodiments, the cutting member 320 can be a metallic whisk of the type shown and described in U.S. Provisional Patent Application Ser. No. 60/816,996, entitled, “Medical Device with Dual Expansion Mechanism,” filed on Jul. 7, 2006, and incorporated herein by reference in its entirety.

In use, the expandable member 310 and the cutting member 320 can be deployed within a patient's body substantially simultaneously. The second shaft 306 can be moved along its longitudinal axis A_{L}, as indicated by arrow B, within the first shaft 304 such that the expandable member 310 and the cutting member 320 can be positioned as desired within a bone structure 10. The bone structure 10 can be, for example, a vertebra. In some embodiments, as described above, the second shaft 306 can be positioned such that the cutting member 320 is positioned within a first portion 12 of the bone structure 10 and the expandable member 310 is positioned within a second portion 14 of the bone structure 10.

Once positioned within the bone structure 10, the cutting member 320 can be actuated by rotating the second shaft 306 with respect to the bone structure 10. In this manner, the cutting member 320 can produce a cutting force on the first portion 12 of the bone structure 10, thereby removing a portion of the first portion 12 of the bone structure 10 to form a first cavity 330, as described above. For example, in some embodiments, the first shaft 304 remains stationary with respect to the bone structure 10 and the second shaft 306 is rotated within the first shaft 304, as indicated by arrow C. The expandable member 310 can then be expanded within the second portion 14 of the bone structure 10, thereby compressing and/or displacing a portion of the second portion 14 of the bone structure 10 to form a second cavity 334, as described above. In this manner, the cutting member 320 and the expandable member 310 can cooperatively form the first cavity 330 and the second cavity 334 without the catheter assembly 300 being removed during the time between the forming of the first cavity 330 and the second cavity 334.

Although the catheter assembly 300 is shown and described in FIG. 8 as including a metallic whisk, in other embodiments, a catheter assembly can include any cutting member suitable for forming a cavity within a bone structure. For example, FIG. 9 shows a portion of a catheter assembly 400 according to an embodiment of the invention that includes a cutting member 420 that includes a cutting element 421. Similar to the catheter assembly 300, the catheter assembly 400 includes a first shaft 404 and a second shaft 406 movably disposed within the first shaft 404. An expandable member 410 is coupled to a distal end portion 414 of the second shaft 406. The cutting member 420 is coupled to an outer surface 415 of the second shaft 406 proximate to the expandable member 410. The cutting element 421 can be, for example, a curette configured to collapse or retract, as shown by the arrow D, to be disposed within the first shaft 404 when not in use.

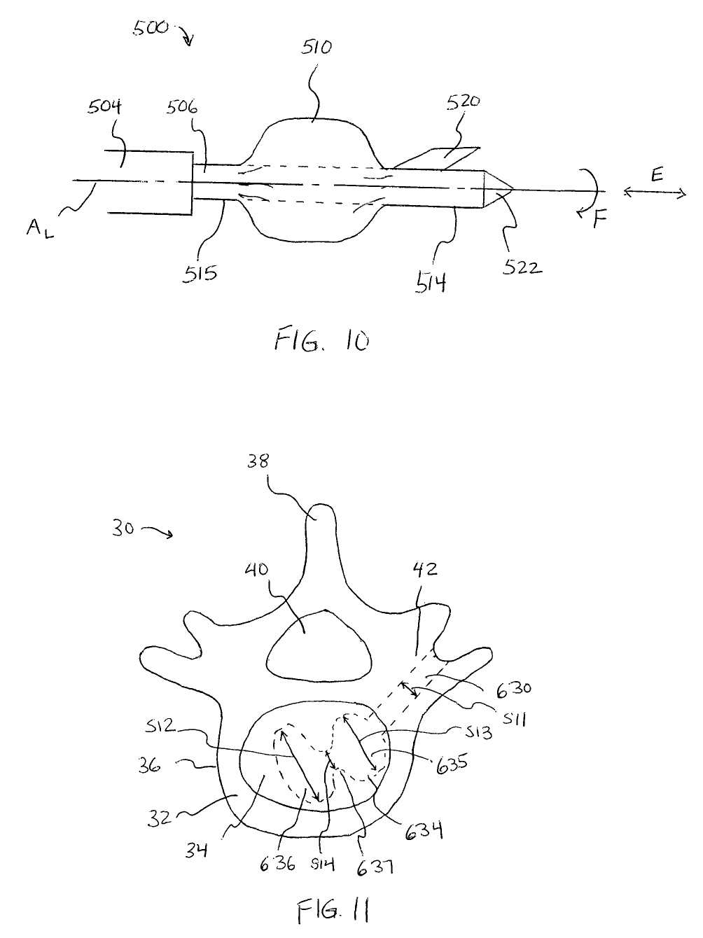

FIG. 10 shows a catheter assembly 500 according to an embodiment of the invention that includes a cutting member 520 disposed at the distal end portion of the catheter assembly 500. The catheter assembly 500 includes a first shaft 504 and a second shaft 506 movably disposed within the first shaft 504, as described above. The second shaft 506 includes a tip portion 522 configured to pierce and/or dilate tissue, a distal end portion 514 and an outer surface 515. In some embodiments, the tip portion 522 can be a trocar tip that is removably disposed within a portion of the second shaft 506. A cutting member 520 of the type shown and described above is coupled to the distal end portion 514 of the second shaft 506. An expandable member 510, of the type shown and described above is coupled to the outer surface 515 of the second shaft 506, proximate to and disposed proximally from the cutting member 520.

As described above, in use, the second shaft 506 can be moved along its longitudinal axis A_{L}, as indicated by arrow E, within the first shaft 504 such that the expandable member 510 and the cutting member 520 can be positioned as desired within a bone structure (not shown in FIG. 10). Once positioned within the bone structure, the cutting member 520 can be actuated to form a cavity within the bone structure by rotating the second shaft 506 within the first shaft 504, as indicated by arrow F. The expandable member 510 can then be expanded to form a cavity within the bone structure.

Although the expandable members are shown and described above as being substantially spherical when in the expanded configuration, in other embodiments, the expandable member can have any suitable shape when in the expanded configuration. For example, in some embodiments, an expandable member can have a shape corresponding to the desired shape of the cavity to be formed by the expandable member. Such shapes can include cylindrical shapes, substantially uniform shapes, irregular shapes, asymmetrical shapes and/or any suitable combination of the shapes disclosed herein.

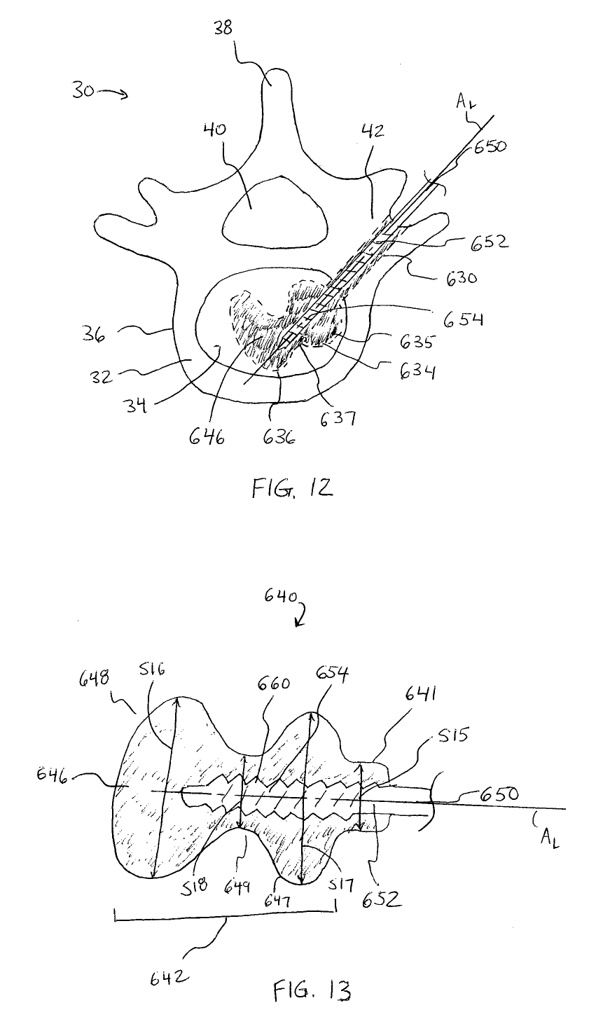

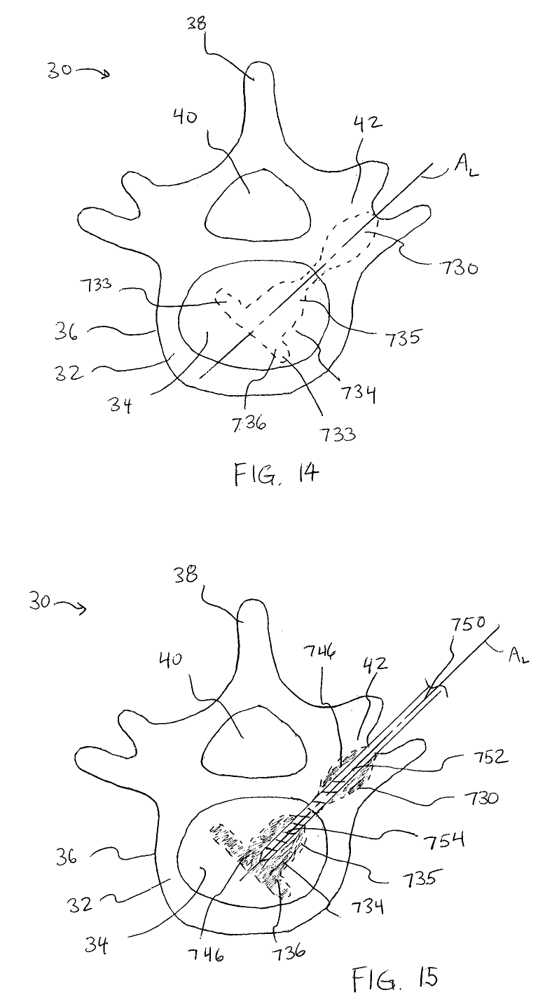

Similarly, although the first cavities, the second cavities and the anchoring members shown and described above have substantially uniform shapes, such as, for example, spheres or cylinders, in some embodiments, the first cavity, the second cavity and/or the anchoring member can have a shape that is non-uniform, irregular and/or asymmetrical. For example, FIG. 11 is a cross-sectional side view from a transverse plane of a vertebra 30 in a first configuration defining a first cavity 630 and a second cavity 634, the second cavity 634 having an irregular shape. The first cavity 630 is defined by at least a portion of the pedicle 42 and/or at least a portion of the outer portion 32 of the vertebral body 36. The first cavity 630 has a substantially cylindrical shape characterized by a size S11 (e.g., the diameter of the cylinder characterizing the first cavity 630). The second cavity 634 is defined by at least a portion of the inner portion 34 of the vertebral body 36 and includes a proximal portion 635, a distal portion 636 and a central portion 637 disposed between the proximal portion 635 and the distal portion 636. More particularly, the distal portion 636 of the second cavity 634 has a size S12 and the proximal portion 635 of the second cavity 634 has a size S13 that is smaller than the size S12. The central portion 637 of the second cavity 634 has a size S14 that is smaller than both size S12 and size S13. In this manner, the second cavity 634 can be shaped to improve the strength of the anchoring member 640 (see FIGS. 12 and 13) to be disposed therein. For example, in some embodiments, the irregular shape of the second cavity 634 increases the surface area of the inner portion 34 of the vertebral body 36 that engages the anchoring member, thereby potentially increasing the pull-out strength of the anchoring member 640.

FIG. 12 is a cross-sectional side view from a transverse plane of the vertebra 30 shown in FIG. 11 in a second configuration, having an anchoring member 640 according to an embodiment of the invention disposed therein. The anchoring member 640 includes a material 646, which can be conveyed into the first cavity 630 and the second cavity 634 as described above, and a fixation device 650. The fixation device 650 is disposed within the vertebra 30 such that a first portion 652 of the fixation device 650 is disposed within the first cavity 630 and in contact with the material 646 contained therein. Similarly, a second portion 654 of the fixation device 650 is disposed within the second cavity 634 and in contact with the material 646 contained therein.

As shown in FIG. 13, the anchoring member 640 has a first end portion 641 and a second end portion 642. The first end portion 641 has a substantially cylindrical shape corresponding to the shape of the first cavity 630 and having a size S15 (e.g., the diameter of the cylinder characterizing the proximal end portion 641). Similarly, the second end portion 642 has an irregular shape corresponding to the shape of the second cavity 634. More particularly, the second end portion 642 of the anchoring member 640 includes a proximal portion 647, a distal portion 648 and a central portion 649 disposed between the proximal portion 647 and the distal portion 648. The distal portion 648 of the second end portion 642 has a size S16, and the proximal portion 647 of the second end portion 642 has a size S17 that is smaller than the size S16. The central portion 649 of the second end portion 642 has a size S18 that is smaller than both size S16 and size S17. As described above, the shapes and/or sizes characterizing the anchoring member 640 can increase its resistance to being pulled out of the vertebra 30. Moreover, as described above, the fixation device 650 can include a series of threads 660 to provide recesses in which the material 646 can flow when in a liquid state, thereby augmenting the strength of the fixation device 650 when the material 646 is in a solid state.

Although the size S12 of the distal portion 636 of the second cavity 634 (and the corresponding size S16) is shown as being larger than the size S13 of the proximal portion 635 of the second cavity (and the corresponding size S17), in other embodiments, the size S12 and/or the size S16 can be smaller than the size S13 and/or the size S17. In yet other embodiments, the size S12 and/or the size S16 can be substantially equal to the size S13 and/or the size S17. Similarly, although the size S14 of the central portion 637 of the second cavity 634 (and the corresponding size S18) is shown as being smaller than either the size S12 or the size S13 (and the corresponding sizes S16 and S17), in some embodiments, the size S14 can larger than or equal to the size S12 and/or the size S13.

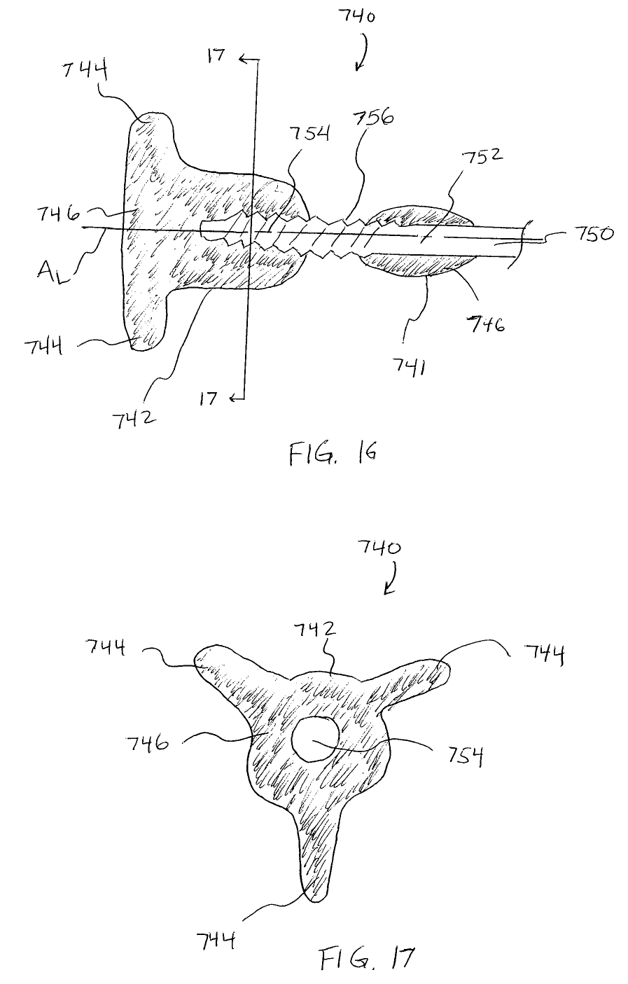

FIGS. 14 and 15 show a cross-sectional side view from a transverse plane of a vertebra 30 in a first configuration and a second configuration respectively, according to another embodiment of the invention. The vertebra 30 defines a first cavity 730 and a second cavity 734 that is spaced apart from the first cavity 730. FIGS. 16 and 17 show an anchoring member 740 according to an embodiment of the invention that corresponds to and is configured to be disposed within the vertebra 30 shown in FIGS. 14 and 15. As described above, the first cavity 730 is defined by at least a portion of the pedicle 42 and/or at least a portion of the outer portion 32 of the vertebral body 36. In contrast to the first cavities shown and described above, the first cavity 730 has an oval-shaped boundary. As previously described, the shape of the first cavity 730 can be configured to increase the surface area of the pedicle 42 that engages the anchoring member 740 (see FIG. 15).

The second cavity 734 is defined by at least a portion of the inner portion 34 of the vertebral body 36 and is spaced apart from the first cavity 730 such that the first cavity 730 and the second cavity 734 are spaced apart and are not contiguous (i.e., the first cavity 730 and the second cavity 734 do not share a common boundary). The second cavity 734 includes a proximal portion 735 and a distal portion 736. The distal portion 736 of the second cavity 734 includes protruding regions 733 that extend radially from a longitudinal axis A_{L} of the second cavity 734. As described above, the size and/or shape of the protruding regions 733 can be configured to increase the strength of an anchoring member 740 (see FIG. 15) disposed within the vertebra 30.

FIG. 15 is a side view of the vertebra 30 shown in FIG. 14 in a second configuration, having an anchoring member 740 according to an embodiment of the invention disposed therein. The anchoring member 740 can be disposed within the vertebra 30 in any suitable manner as described herein. The anchoring member 740 includes a material 746 and a fixation device 750. The anchoring member 740 can be conveyed into the first cavity 730 and the second cavity 734 in a substantially liquid state, as described above. The fixation device 750 is disposed within the vertebra 30 such that a first portion 752 of the fixation device 750 is disposed within the first cavity 730 and in contact with the material 746 contained therein. Similarly, a second portion 754 of the fixation device 750 is disposed within the second cavity 734 and in contact with the material 746 contained therein.

As shown in FIGS. 16 and 17, the anchoring member 740 has a first end portion 741 and a second end portion 742. The first end portion 741 has an oval-shaped boundary corresponding to the shape of the first cavity 730. Similarly, the second end portion 742 has protruding portions 744 corresponding to the protruding regions 733 of the second cavity 734. As shown in FIG. 17, the protruding portions 744 extend radially from the longitudinal axis A_{L} of the anchoring member 740. In this manner, the protruding portions 744 can augment the anchoring member 740 to increase its resistance to rotation about the longitudinal axis A_{L}. The protruding portions 744 can extend from the second end portion 742 of the anchoring member 740 in any suitable fashion. In some embodiments, for example, the protruding portions 744 can extend substantially symmetrically about the longitudinal axis A_{L}. In other embodiments, the protruding portions 744 can extend substantially asymmetrically about the longitudinal axis A_{L}. In yet other embodiments, the protruding portions 744 can extend from the second end portion 742 in a direction that is not substantially normal to the longitudinal axis A_{L}. Moreover, in some embodiments, the protruding portions 744 can have different shapes and/or sizes.

As shown in FIG. 16, the material 746 included in the first end portion 741 of the anchoring member 740 is spaced apart from the material 746 included in the second end portion 742 of the anchoring member 740. Said another way, the fixation device 750 includes a third portion 756 disposed between the first portion 752 and the second portion 754 that is substantially devoid of the material 746.

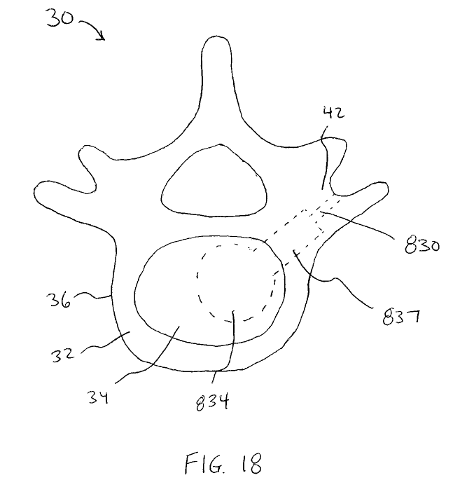

Although the bone structures shown and described above define a first cavity and a second cavity, in some embodiments, a bone structure can define any number of cavities. Similarly, an anchoring member can include any number of portions corresponding to the cavities defined by the bone structure. For example, FIG. 18 shows a vertebra 30 according to an embodiment of the invention defining a first cavity 830, a second cavity 834 and a third cavity 837, each being mutually exclusive of the others. As described above, the first cavity 830 can be defined by at least a first portion of the vertebra 30 (e.g., the first portion of the vertebra 30 can include the pedicle 42). The second cavity 834 can be defined by at least a second portion of the vertebra 30 (e.g., the second portion of the vertebra 30 can include the inner portion 34). The third cavity 837 can be defined by at least a third portion of the vertebra 30 (e.g., the third portion of the vertebra 30 can include portions of the inner portion 34 and/or the outer portion 32). As shown, the first cavity 830, the second cavity 834 and the third cavity 837 are each mutually exclusive of the others.

Similar to the medical devices described above, and using methods similar to those described above, an augmentation material, such as a bone cement, can be conveyed into the first cavity 830, the second cavity 834 and the third cavity 837. A fixation device (not shown in FIG. 18) can then be disposed within the vertebra 30 such that a first portion of the fixation device is within the first cavity 830 and in contact with the augmentation material, a second portion of the fixation device is within the second cavity 830 and in contact with the augmentation material and a third portion of the fixation device is within the third cavity 830 and in contact with the augmentation material. In this manner, the resulting anchoring member (not shown in FIG. 18) can conform to the shape and size of the first cavity 830, the second cavity 834 and the third cavity 837 to increase the strength of the fixation device within the vertebra.

While various embodiments of the invention have been described above, it should be understood that they have been presented by way of example only, and not limitation. Where methods described above indicate certain events occurring in certain order, the ordering of certain events may be modified. Additionally, certain of the events may be performed concurrently in a parallel process when possible, as well as performed sequentially as described above. Thus, the breadth and scope of the invention should not be limited by any of the above-described embodiments. While the invention has been particularly shown and described with reference to specific embodiments thereof, it will be understood that various changes in form and details may be made.

For example, in some embodiments, the methods shown and described above include certain events that occur during the same surgical procedure. For example, in some embodiments, a method includes forming a first cavity, forming a second cavity, conveying a material to the first cavity and the second cavity, and inserting a fixation device, as described above, during the same surgical procedure.

Although the material used to augment the fixation device within the first cavity is shown and described as being the same material used to augment the fixation device within the second cavity, in some embodiments, a first material can be used to augment the fixation device within the first cavity and a second material, different than the first material, can be used to augment the fixation device within the second cavity. For example, in some embodiments, a first type of bone cement can be used within the first cavity and a second, different type of bone cement can be used in within the second cavity. Moreover, the augmentation material is not limited to bone cements, but can be any suitable compound, including bone growth enhancers or the like.

Although the fixation devices are shown and described as being substantially solid member, in other embodiments, a fixation device can include perforations or openings configured to receive a portion of the augmentation material to further increase the strength of the resulting anchoring member. In other embodiments, a fixation device can have a hollow portion configured to receive a portion of the augmentation material and/or any new bone growth therein.

Although the fixation devices are shown and described as including threads to provide recesses in which an augmentation material can flow when in a liquid state, in other embodiments, a fixation device can include any type of protrusion suitable for increasing the surface area of the fixation device and/or the area of contact between the fixation device and the augmentation material. In some embodiments, for example, the protrusions can be shaped to increase the pull-out strength of the anchoring member and/or increase the resistance of the fixation device to being rotated about its longitudinal axis. For example, a protrusion configured to increase the pull-out strength of the anchoring member can have a surface that extends radially from the fixation device and is substantially normal to the longitudinal axis of the fixation device (threads are one such example). In other embodiments, a protrusion configured to increase the resistance of the fixation device to being rotated about its longitudinal axis can have a surface that extends radially from the fixation device and is substantially normal to the longitudinal axis of the fixation device. In yet other embodiments, a protrusion configured to increase the resistance of the fixation device to being rotated normal to its longitudinal axis can have a surface that extends in any suitable direction from the fixation device.

Although the anchoring members shown and described above are characterized by longitudinal axis, in other embodiments an anchoring member can be curved in the longitudinal direction. Similarly, although the fixation devices are shown and described as being substantially linear, in some embodiments, a fixation device can have any suitable shape. For example, in some embodiments, a fixation device can have two discontinuous linear portions that form a “V” shape, an “X” shape or the like.

Although the fixation devices and/or anchoring members are shown and described above as having at least one end portion that extends from a bone structure after the fixation device and/or anchoring member has been disposed therein, in some embodiments, a fixation device and/or anchoring member can be disposed within a bone structure such that no portion of the fixation device and/or anchoring member extends from the bone structure. In other embodiments, a fixation device and/or anchoring member can be disposed within a bone structure such that the fixation device and/or anchoring member extends from two or more sides of the bone structure.

Although the catheter assembly is shown and described as being used by first actuating the cutting member to form the first cavity and followed by actuating the expandable member to form the second cavity, in some embodiments, a catheter assembly can be used by actuating the expandable member first, followed by actuating the cutting member. In other embodiments, a catheter assembly can be used by actuating the expandable member and the cutting member substantially simultaneously.

Similarly, although the methods shown and described above include conveying a material to a first cavity and a second cavity before disposing a fixation device within the first cavity and second cavity, in some embodiments, a method can include disposing a fixation device within the first cavity and second cavity, followed by conveying the material to the first cavity and the second cavity. In some embodiments, for example, the fixation device can be used to convey the material into the first cavity and/or the second cavity.

Although the methods are shown and described above as including removing the cavity-forming tool before conveying the material to the first cavity and/or the second cavity, in some embodiments, a method can include conveying the material to the first cavity and/or the second cavity without first removing the cavity-forming tool. In some embodiments, for example, the cavity-forming tool can include delivery lumen configured to convey the material to the first cavity and/or the second cavity.

Although the methods are shown and described above as including forming at least a first cavity and a second cavity using a cavity-forming tool that engages a portion of the bone structure to remove, displace and/or compress a portion of the bone structure, in some embodiments, a method can include forming a cavity within a bone structure using a cavity-forming tool that does not directly engage the portion of the bone structure in which the cavity is formed. For example, in some embodiments, a method can include forming a first cavity using a cavity-forming tool that removes a first portion of a bone structure. The method can then include forming a second cavity by conveying a viscous material into a second portion of the bone structure. The viscous material can be conveyed into the second portion of the bone structure at a pressure sufficient to compress and/or displace the second portion of the bone structure, thereby forming the second cavity. Moreover, the viscous material can any type of material formulated to augment a fixation device, as described above. Said another way, in some embodiments, a method can include forming a cavity and conveying a material into the cavity substantially simultaneously. Moreover, in some embodiments, a method can include forming a cavity and conveying a material into the cavity using a single tool.

Similarly, although the catheter assembly is shown and described as including a cutting member configured to form a first cavity and an expandable member configured to form a second cavity, in some embodiments, a catheter assembly can include two cutting members (e.g., a curette and a whisk) configured to form the first cavity and the second cavity. In other embodiments, a catheter assembly can include two expandable members configured to form the first cavity and the second cavity. In yet other embodiments, a catheter assembly can include a cutting member configured to form a first cavity and a delivery lumen configured to convey a material and form a second cavity, as described above.

Although various embodiments have been described as having particular features and/or combinations of components, other embodiments are possible having a combination of any features and/or components from any of embodiments as discussed above. For example, one such embodiment includes a catheter having a first cutting member that includes a metallic whisk, a second cutting member that includes a curette and an expandable member.

What is claimed is:

1. An apparatus, comprising:
an expandable member configured to form a cavity within a cancellous portion of a bone; and
a cutting member configured to cut a cortical portion of the bone, the cutting member being coupled to the expandable member.

2. The apparatus of claim 1, wherein the expandable member and the cutting member are configured to be deployed within a body substantially simultaneously.

3. The apparatus of claim 1, wherein the expandable member and the cutting member are configured to be actuated cooperatively.

4. The apparatus of claim 1, wherein the expandable member and the cutting member are configured to be actuated serially.

5. The apparatus of claim 1, wherein the cutting member includes a whisk.

6. The apparatus of claim 1, wherein the cutting member includes a curette.

7. The apparatus of claim 1, further comprising:
a first shaft; and
a second shaft movably disposable within the first shaft, the cutting portion being coupled to an outer surface of the second shaft, the expandable member being coupled to the second shaft.

8. The apparatus of claim 1, further comprising:
a first shaft; and
a second shaft, a portion of which is rotatably disposable within the first shaft, a portion of the cutting member being coupled to an outer surface of the second shaft such that the cutting member produces a cutting force on the cortical portion of the bone of when the second shaft is rotated within the first shaft and the cutting member is in contact with the cortical portion of the bone.

9. The apparatus of claim 1, wherein the bone is a vertebra.

10. The apparatus of claim 1, wherein the expandable member is configured to have a substantially spherical shape when expanded.

11. The apparatus of claim 1, wherein a distal portion of the expandable member is configured to have a size greater than a size of a proximal portion of the expandable member when the expandable member is expanded.

12. The apparatus of claim 1, wherein the expandable member has proximal portion, a distal portion and a central portion disposed between the proximal portion and the distal portion, the central portion configured to have a size smaller than a size of each of the proximal portion and the distal portion when the expandable member is expanded.

13. The apparatus of claim 1, wherein the expandable member is configured to have an asymmetrical shape when expanded.

14. The apparatus of claim 1, wherein a proximal end portion of the expandable member is disposed between a distal end portion of the expandable member and the cutting member.

15. A method, comprising:
forming a first cavity within a first portion of a bone;
forming a second cavity within a second portion of the bone, the first portion and the second portion being mutually exclusive;
conveying a material to the first cavity and the second cavity; and
inserting a fixation device into the bone such that a first portion of the fixation device is disposed within the first cavity and in contact with the material and a second portion of the fixation device is disposed within the second cavity and in contact with the material.

16. The method of claim 15, wherein a size of the second cavity is larger than a size of the first cavity.

17. The method of claim 15, wherein a shape of the second cavity is different than a shape of the first cavity.

18. The method of claim 15, wherein the second cavity has a distal portion and a proximal portion, a size of the distal portion being greater than a size of the proximal portion.

19. The method of claim 15, wherein a shape of the second cavity is asymmetrical.

20. The method of claim 15, wherein the first cavity is contiguous with the second cavity.

21. The method of claim 15, wherein the first portion of the bone has a characteristic different than a characteristic of the second portion of the bone.

22. The method of claim 15, wherein:
the first portion of the bone has a bone density; and
the second portion of the bone has a bone density different than the bone density of the first portion of the bone.

23. The method of claim 15, wherein:
the first portion of the bone includes cortical bone; and
the second portion of the bone includes cancellous bone.

24. The method of claim 15, wherein the bone is a vertebra.

25. The method of claim 15 at least one of the forming the first cavity, the forming the second cavity, the conveying the material or the inserting the fixation device is done percutaneously.

26. The method of claim 15, wherein at least one of the forming the first cavity, the forming the second cavity, the conveying the material or the inserting the fixation device is done through a cannula.

27. The method of claim 15, wherein:
the forming the first cavity and the forming the second cavity are done with a tool configured to be disposed within a cannula; and
the tool is not removed from the cannula during a time period between the forming the first cavity and the forming the second cavity.

28. The method of claim 15, wherein the forming the first cavity includes removing a portion of the first portion of the bone.

29. The method of claim 15, wherein the forming the second cavity includes compressing a portion of the second portion of the bone.

30. The method of claim 15, wherein the forming the second cavity includes compressing a portion of the bone via an expandable member.

31. The method of claim 15, wherein the material is a liquid configured to harden after being conveyed into the first cavity and the second cavity.

32. The method of claim 15, wherein:
the fixation device is a pedicle screw; and
the material is configured to augment the pedicle screw.

33. The method of claim 15, wherein:
the fixation device is a pedicle screw; and
the material is a bone cement.

34. The method of claim 15, further comprising:
forming a third cavity within a third portion of the bone, the third portion being mutually exclusive from the first portion and the second portion;
conveying the material to the third cavity; and
inserting the fixation device into the bone such that a third portion of the fixation device is disposed within the third cavity.

35. The method of claim 15, further comprising:
forming a third cavity within a third portion of the bone, the third portion being mutually exclusive from the first portion and the second portion, the third cavity being contiguous with the first cavity;
conveying the material to the third cavity; and
inserting the fixation device into the bone such that a third portion of the fixation device is disposed within the third cavity.

36. The method of claim 15, further comprising:
forming a third cavity within a third portion of the bone, the third portion being mutually exclusive from the first portion and the second portion, the third cavity being contiguous with the second cavity;
conveying the material to the third cavity; and
inserting the fixation device into the bone such that a third portion of the fixation device is disposed within the third cavity.

37. An apparatus, comprising:
an anchoring member configured to limit the movement of a pedicle screw within a vertebra, a distal portion of the anchoring member having a size greater than a size of a proximal portion of the anchoring member.

38. The apparatus of claim 37, wherein the anchoring member has a first configuration in which a portion of the anchoring member is substantially liquid and a second configuration in which the portion of the anchoring member is substantially solid, the anchoring member configured to limit the movement of the pedicle screw when in the second configuration.

39. The apparatus of claim 37, wherein:
the distal portion of the anchoring member has a substantially spherical shape; and
the proximal portion of the anchoring member has a substantially cylindrical shape.

40. The apparatus of claim 37, wherein the distal portion of the anchoring member has at least two protrusions extending radially from a longitudinal axis of the anchoring member.

41. The apparatus of claim 37, wherein the anchoring member includes a bone cement.

42. The apparatus of claim 37, further comprising the pedicle screw.

43. A kit, comprising:
a catheter having an expandable member configured to form a cavity within a cancellous portion of a bone and a cutting member configured to cut a cortical portion of the bone, the cutting member being disposed proximate to the expandable member; and
a cannula configured be inserted percutaneously, the cannula being configured to receive a portion of the catheter.

44. The kit of claim 43, further comprising a bone cement.

45. The kit of claim 43, further comprising a pedicle screw.

46. An apparatus, comprising:
a portion of a cortical bone defining a first cavity;
a portion of a cancellous bone defining a second cavity, the second cavity being longitudinally displaced from the first cavity;
an anchoring member having a first portion and a second portion, the first portion disposed within the first cavity, the second portion disposed within the second cavity; and
a fastener coupled to the anchoring member.

47. The apparatus of claim 46, wherein:
the second cavity has a size greater than a size of the first cavity;
the first portion of the anchoring member has a size corresponding to the size of the first cavity; and
the second portion of the anchoring member has a size corresponding to the size of the second cavity.

48. The apparatus of claim 46, wherein:
the first cavity has a shape;
the first portion of the anchoring member has a shape corresponding to the shape of the first cavity;
the second cavity has a shape; and
the second portion of the anchoring member has a shape corresponding to the shape of the second cavity.

49. The apparatus of claim 46, wherein the fastener is a pedicle screw.

50. The apparatus of claim 46, wherein:
a first portion of the fastener is disposed within the first portion of the anchoring member; and
a second portion of the fastener is disposed within the second portion of the anchoring member.
